SEQUENCE LISTING (2 sequences)

SEQ ID NO: 1 - DNA, 1881 bp, Artificial Sequence
  source 1..1881: /mol_type=“DNA” /note=“recombinant nucleic acid molecule” /organism=“Artificial Sequence”
  source 1..1881: /mol_type=“DNA” /note=“CDS comprising a phagolysosomal domain (211-1722nt) and a stop codon (1879-1881)” /organism=“Artificial Sequence”
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcagtctgcaaagcaatccgctgcaaataaattgcactcagcaggacaaagcacgaaagatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaatatgcattagcctatggaagtcagggtgatcttaatccattaattaatgaaatcagcaaaatcatttcagctgcagttctttcctctttaacatcgaagctacctgcagagttcgttaggcgcggatccggaattcgaagcttatcgatgtcgacgtag

SEQ ID NO: 2 - protein, 626 aa, Artificial Sequence
  SOURCE 1..626: /mol_type=“protein” /note=“CORRESPONDING AMINO ACID SEQUENCE OF SEQ ID NO 1.” /organism=“Artificial Sequence”
MTDVSRKIRAWGRRLMIGTAAAVVLPGLVGLAGGAATAGAFSRPGLPVEYLQSAKQSAANKLHSAGQSTKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIEYALAYGSQGDLNPLINEISKIISAAVLSSLTSKLPAEFVRRGSGIRSLSMST

(19) United States
(12) Patent Application Publication
(10) Pub. No.: US 2022/0160858 A1
(43) Pub. Date: May 26, 2022

(54) Recombinant Mycobacterium as an Immunotherapeutic Agent for the Treatment of Cancer

(71) Applicant: VAKZINE PROJEKT MANAGEMENT GMBH, Hannover (DE)

(72) Inventor: Leander GRODE, Braunschweig (DE)

(73) Assignee: VAKZINE PROJEKT MANAGEMENT GMBH, Hannover (DE)

(21) Appl. No.: 17/667,784

(22) Filed: Feb. 9, 2022

Related U.S. Application Data
(63) Continuation-in-part of application No. 16/688,371, filed on Nov. 19, 2019.
Application No. 16/688,371 is a division of application No. 15/571,415, filed on Nov. 2, 2017, now Pat. No. 10,525,118.
(60) Provisional application No. 62/387,407, filed on Dec. 23, 2015.

(30) Foreign Application Priority Data
May 4, 2015 (EP) 15166206.1

(51) Int. Cl. A61K 39/04; A61P 13/10; A61P 35/00

(52) U.S. Cl. CPC A61K 39/04; A61K 2039/522; A61P 35/00; A61P 13/10

(57) ABSTRACT

The invention relates to a recombinant Mycobacterium cell for use as an immunotherapeutic agent in the treatment of cancer, particularly in the treatment of solid tumors. More particularly, the invention relates to the immunotherapy of bladder carcinoma.

### CROSS REFERENCE TO RELATED APPLICATION

This is a continuation-in-part of U.S. Ser. No. 16/688,371 filed Nov. 19, 2019, which is a divisional of U.S. Ser. No. 15/571,415 filed Nov. 2, 2017, now U.S. Pat. No. 10,525,118 issued on Jan. 7, 2020, which is a 35 U.S.C. 371 National Phase Entry Application from PCT/EP2016/059872, filed May 3, 2016, which claims the benefit of provisional application No. 62/387,407, filed Dec. 23, 2015, which claims the benefit of European Patent Application No. 15166206.1 filed on May 4, 2015, the disclosures of which are incorporated herein in their entirety by reference.

The instant application contains a Sequence Listing which has been submitted via EFS-Web and is hereby incorporated by reference in its entirety. Said Sequence_Listing, created on Feb. 7, 2022, is named 60182PUSW02_Sequence_Listing.txt and is 9 kilobytes in size.

### DESCRIPTION

The invention relates to a recombinant Mycobacterium cell for use as an immunotherapeutic agent in the treatment of cancer, particularly in the treatment of solid tumors. More particularly, the invention relates to the immunotherapy of bladder carcinoma.

Urothelial bladder carcinoma is the 5th most common cancer. In the United States, about 75.000 new cases are diagnosed each year 4.5% of all new cancers, and approximately 15.600 deaths are expected. In Germany, about 16.000 new cases are diagnosed each year. Because a recurrence of disease is likely in bladder carcinoma, patients must undergo surveillance for an extended period.

Most bladder carcinomas begin in transitional epithelial cells that make up the inner lining of the bladder. As these tumors grow, they can invade the surrounding connective tissue and muscle. In advanced disease, tumors spread beyond the bladder to nearby lymph nodes or pelvic organs or metastasize to more distant organs such as lung, liver and bone.

The overall 5-year survival rate for bladder carcinoma is 77%, and this rate has not changed significantly over the last 10 years. When considered by stage, the 5-year relative survival rates for patients with tumors restricted to the inner layer of the bladder are 96% and 69%, respectively. The rates drop to 34% for those with disease that has spread locally beyond the bladder and to 6% with distant metastases.

For patients with non-muscle invasive bladder carcinoma, treatment usually involves a surgical removal of the tumor followed by chemotherapy, usually mitomycin C, within the bladder (so-called intravesical chemotherapy). After recovering from surgery, patients with a lower risk of disease progression may undergo surveillance or additional intravesical chemotherapy. Patients with moderate-to-high-grade disease often receive intravesical immunotherapy with an attenuated live bacterium Bacillus Calmette Guerin (BCG). BCG was the first FDA-approved immunotherapy and helps reduce the risk of bladder carcinoma recurrence by stimulating an immune response that targets the bacteria as well as any bladder carcinoma cells. In some patients, however, conventional BCG therapy has found to be less effective, particularly after repeated administration. The terms “conventional BCG therapy” and “standard BCG therapy” may be used interchangeably herein.

Standard treatment for patients with muscle-invasive bladder carcinoma includes cisplatin-based chemotherapy followed by surgical removal of the bladder or radiation therapy and concomitant chemotherapy. Recurrent bladder carcinoma may be treated with combination therapy regimens, including gemcitabine plus cisplatin or methotrexate, vinblastine, doxorubicin plus cisplatin.

In the treatment of bladder carcinoma, tumor recurrence is a major concern, even for patients with low-grade disease and requires extensive follow-up. Better treatments, such as novel immunotherapies, might reduce recurrence rates and improve the survival of patients with bladder carcinoma.

A recombinant BCG strain expressing a phagolysosomal escape domain is described in WO 99/101496, the content of which is herein incorporated by reference. The phagolysosomal escape domain enables the strain to escape from the phagosome of infected host cells by perforating the membrane of the phagosome. In order to provide an acidic phagosomal pH for optimal phagolysosomal escape activity, a urease-deficient recombinant strain was developed. This strain is disclosed in WO 2004/094469, the content of which is herein incorporated.

WO 2012/085101, the content of which is herein incorporated, discloses that a recombinant BCG strain expressing membrane-perforating listeriolysin (Hly) of Listeria monocytogenes and devoid of urease C induces superior protection against aerogenic challenge with Mycobacterium tuberculosis (Mtb) as compared to parental BCG in a preclinical model. Further, it is shown that both the recombinant and the parenteral strain induce marked Th1 immune responses, whilst only the recombinant BCG strain elicits are profound Th17 response in addition.

In the present study, it was found that a recombinant urease-deficient and listeriolysin-expressing recombinant BCG strain induces a superior immune response compared to the parenteral BCG in an animal model.

A subject-matter of the present invention is a recombinant Mycobacterium cell which comprises a recombinant nucleic acid molecule encoding a fusion polypeptide comprising:
(a) a domain capable of eliciting an immune response, and
(b) a phagolysosomal escape domain

for use as an immunotherapeutic agent in the treatment in the treatment of bladder carcinoma, in particular recurrent bladder carcinoma, wherein the individual to be treated has relapsed and/or has progressed after a first treatment of bladder carcinoma.

A further aspect of the present invention is a method for the immunotherapeutic treatment of solid tumors in a subject in need thereof, comprising administering to said subject a recombinant Mycobacterium cell which comprises a recombinant nucleic acid molecule encoding a fusion polypeptide comprising:

(a) a domain capable of eliciting an immune response, and

(b) a phagolysosomal escape domain.

According to the present invention it was found that vesicular instillation of a recombinant BCG cell into the bladder of rats surprisingly results in an increased infiltration of urinary bladder tissue by lymphocytes, particularly CD4- and CD8-positive lymphocytes resulting in a high incidence of focal and/or multifocal lymphocytic infiltration. In contrast thereto, the urinary bladder tissue of animals treated with standard BCG showed CD4- and CD8-positive lymphocytes only as single cell infiltrate levels (diffuse infiltration). Further, administration of a recombinant BCG cell did not raise any safety issues.

Presently, a phase I/II clinical trial assessing safety and efficacy of intravesical instillation of recombinant BCG in human patients with recurrent non-muscle invasive bladder carcinoma after standard BCG therapy is conducted.

Thus, the present invention also relates to a recombinant Mycobacterium cell as described above for use as an immunotherapeutic agent in the treatment of solid tumors in order to obtain focal and/or multifocal lymphocytic infiltration, e.g. with CD4 and CD8 T cells at the site of administration. The recombinant Mycobacterium cell of the present invention is particularly suitable for use in human medicine.

The immunotherapeutic agent is a live recombinant Mycobacterium cell which comprises a recombinant nucleic acid molecule encoding a fusion polypeptide comprising (a) a domain capable of eliciting an immune response and (b) a phagolysosomal escape domain. The domain capable of eliciting an immune response is preferably an immunogenic peptide or polypeptide from a pathogen or an immunogenic fragment thereof.

The Mycobacterium cell is preferably an M. bovis cell, an M. tuberculosis cell, particularly an attenuated M. tuberculosis cell or other Mycobacteria, e.g. M. microti, M. smegmatis, M. canettii, M. marinum or M. fortuitum. More preferably, the cell is an attenuated recombinant M. bovis (BCG) cell, particularly an M. bovis BCG cell, more particularly a recombinant M. bovis BCG cell from strain Danish subtype Prague (Brosch et al., Proc. Natl. Acad. Sci. USA, 104 (2007), 5396-5601). In an especially preferred embodiment, the Mycobacterium cell is recombinant urease-deficient. In an especially preferred embodiment the ureC sequence of the Mycobacterium cell is inactivated (ΔUrec), e.g. by constructing a suicide vector containing a ureC gene disrupted by a selection marker gene, e.g. the hygromycin gene, transforming the target cell with the vector and screening for selection marker-positive cells having a urease negative phenotype. In an even more preferred embodiment, the selection marker gene, i.e. the hygromycin gene, is subsequently inactivated. In this embodiment, the cell is a selection marker-free recombinant Mycobacterium cell. Most preferably, the cell is selection marker-free recombinant BCG strain Danish subtype Prague characterized as recombinant BCG ΔUrec::Hly+.

The domain capable of eliciting an immune response is preferably selected from immunogenic peptides or polypeptides from M. bovis, M. tuberculosis or M. leprae or from immunogenic fragments thereof having a length of at least 6, preferably at least 8 amino acids, more preferably at least 9 amino acids and e.g. up to 20 amino acids. Specific examples for suitable antigens are Ag85B (p30) from M. tuberculosis, Ag85B (α-antigen) from M. bovis BCG, Ag85A from M. tuberculosis and ESAT-6 from M. tuberculosis and fragments thereof. In other embodiments, the domain capable of eliciting an immune response is selected from non-Mycobacterium polypeptides.

More preferably, the immunogenic domain is derived from the antigen Ag85B. Most preferably, the immunogenic domain comprises the sequence from aa.41 to aa.51 in SEQ ID No.2.

The recombinant nucleic acid molecule further comprises a phagolysosomal escape domain, i.e. a polypeptide domain which provides for an escape of the fusion polypeptide from the phagolysosome into the cytosol of mammalian cells. Preferably, the phagolysosomal escape domain is a Listeria phagolysosomal escape domain, which is described in U.S. Pat. No. 5,733,151, herein incorporated by reference. More preferably, the phagolysosomal escape domain is derived from the listeriolysin gene (Hly) of L. monocytogenes. Most preferably, the phagolysosomal domain is encoded by a nucleic acid molecule selected from: (a) a nucleotide sequence comprising nucleotides 211-1722 as shown in SEQ ID No.1, (b) a nucleotide sequence which encodes the same amino acid sequence as the sequence from (a), and (c) a nucleotide sequence hybridizing under stringent conditions with the sequence from (a) or (b).

Apart from the nucleotide sequence depicted in SEQ ID No.1 the present invention also comprises nucleic acid sequences hybridizing therewith. In the present invention the term “hybridization” is used as defined in Sambrook et al. (Molecular Cloning. A laboratory manual, Cold Spring Harbor Laboratory Press (1989), 1.101-1.104). In accordance with the present invention the term “hybridization” is used if a positive hybridization signal can still be observed after washing for one hour with 1×SSC and 0.1% SDS at 55° C., preferably at 62° C. and more preferably at 68° C., particularly for 1 hour in 0.2×SSC and 0.1% SDS at 55° C., preferably at 62° C. and more preferably at 68° C. A sequence hybridizing with a nucleotide sequence as per SEQ ID No.1 under such washing conditions is a phagolysosomal escape domain encoding nucleotide sequence preferred by the subject invention.

A nucleotide sequence encoding a phagolysosomal escape domain as described above may be directly obtained from a Listeria organism or from any recombinant source e.g. a recombinant E. coli cell containing the corresponding Listeria nucleic acid molecule or a variant thereof as described above.

Preferably, the recombinant nucleic acid molecule encoding for a fusion polypeptide contains a signal peptide encoding sequence. More preferably, the signal sequence is a signal sequence active in Mycobacteria, preferably in M.bovis, e.g. a native M.bovis signal sequence. A preferred example of a suitable signal sequence is the nucleotide sequence coding for the Ag85B signal peptide which is depicted in SEQ ID No.1 from nucleotide 1 to 120.

Further, it is preferred that a peptide linker be provided between the immunogenic domain and the phagolysosomal escape domain. Preferably, said peptide linker has a length of from 5 to 50 amino acids. More preferably, a sequence encoding a linker as shown in SEQ ID No.1 from nucleotide 154 to 210 or a sequence corresponding thereto as regards the degeneration of the genetic code.

The nucleic acid may be located on a recombinant vector. Preferably, the recombinant vector is a prokaryotic vector, i.e. a vector containing elements for replication or/and genomic integration in prokaryotic cells. Preferably, the recombinant vector carries the nucleic acid molecule of the present invention operatively linked with an expression control sequence. The expression control sequence is preferably an expression control sequence active in Mycobacteria, particularly in M.bovis. The vector can be an extrachromosomal vector or a vector suitable for integration into the chromosome. Examples of such vectors are known to the man skilled in the art and, for instance, given in Sambrook et al. supra.

The immunotherapeutic agent of the present invention is suitable for the treatment of solid tumors, such as bladder, lung, liver, breast, kidney or prostate tumors. Particularly, the present invention is suitable for the treatment of non-invasive solid tumors. In an especially preferred embodiment, the solid tumor is bladder carcinoma, e.g., non-invasive bladder carcinoma, e.g. non-invasive papillary carcinoma in situ (Tₐ), non-invasive carcinoma in situ (T_{cis}), tumor invading subepithelial connective tissue (T₁), tumor invading superficial muscle (inner half) (T₂ₐ), tumor invading deep muscle (outer half) (T_{2b}), tumor invading perivesical tissue (T₃ including T₃ₐ and T_{3b}), tumor invading prostate, uterus or vagina (T₄ₐ), and tumor invading pelvic wall or abdominal wall (T_{4b}). Particularly, the tumor is a superficial tumor or carcinoma in situ (T_{cis}), non-invasive papillary carcinoma (Tₐ), or a tumor invading subepithelial connective tissue (T₁). The immunotherapeutic treatment is suitable for the treatment of primary tumors and/or for the treatment of recurring tumors.

The immunotherapeutic agent is preferably locally administered to the tumor site, i.e., to the site of a primary tumor before surgery or after surgery and optionally after chemotherapy. For the treatment of urothelial bladder carcinoma, the agent is preferably administered by vesicular instillation into the urinary bladder. For other tumors, the administration may involve local injection or, in case of lung tumors, pulmonal administration.

The immunotherapeutic agent of the invention may be administered as a first-line immunotherapy in patients, who have not been treated previously with an anti-tumor-immunotherapeutic agent such as standard BCG, or a follow-up immunotherapy in patients who have been treated previously with anti-tumor-immunotherapeutic agent such as standard BCG. The immunotherapeutic agent may be administered with a newly diagnosed solid tumor, e.g., a bladder carcinoma, or to patients, particularly patients with recurrent solid tumors, e.g., bladder carcinoma.

According to a preferred embodiment of the invention the immunotherapeutic is administered as a second-line treatment. According to such embodiments, the individual to be treated may have received a first treatment, in particular of bladder carcinoma treatment, selected from the group of cisplatin-based chemotherapy, in particular cisplatin-based chemotherapy followed by surgical removal of the bladder or radiation therapy, concomitant chemotherapy and standard BCG. Preferably, the individual to be treated has received standard BCG as a first treatment of bladder carcinoma and/or underwent cystectomy or another local treatment or systemic chemotherapy. The bladder carcinoma to be treated is preferably a non-muscle-invasive bladder cancer (NMIBC). According to an especially preferred embodiment, the individual to be treated is a smoker.

The immunotherapeutic agent is administered to the subject to be treated in an effective dose. For a human subject, the dose for an administration may be about 10⁶ to 10¹⁰ viable units (CFU), e.g. about 10⁷ to 10⁹ or 10⁸ to 10⁹ viable units. Preferably, the immunotherapeutic agent is administered several times, e.g. at least 3 times or at least 5 times up to 30 times, particularly about 15 times, at predetermined times during the treatment.

The immunotherapeutic agent is usually provided as a pharmaceutical preparation which comprises the recombinant Mycobacterial cell in solid form, e.g., a lyophilized or cryoconserved preparation, which is reconstituted with a suitable liquid carrier before use. Alternatively, the preparation may be provided in liquid form, e.g., as suspension.

In one embodiment, the immunotherapeutic agent of the invention is administered for the treatment of carcinoma in situ. A standard schedule may comprise weekly administration of the agent for at least 4, e.g., 4, 5, 6, 7 or 8 weeks as an induction therapy. The induction therapy should not start until 2-3 weeks after primary tumor surgery. After a treatment-free interval of, e.g., 4 weeks, administration may continue using maintenance therapy for at least 6 months or at least 1 year.

In a further embodiment, the immunotherapeutic agent is administered in an induction therapy in the prophylactic treatment of tumor recurrence. In this embodiment, therapy may start about 2-3 weeks after biopsy of the tumor site and be repeated, e.g., at weekly intervals for at least 4, e.g., 4, 5, 6, 7 or 8 weeks. In intermediate and high-risk tumors this may be followed by maintenance therapy.

Maintenance therapy may comprise long-term therapy, e.g., 6, 9 or 12 months therapy or even longer with treatments at monthly intervals. Alternatively, maintenance therapy may comprise 2, 3 or 4 administrations at weekly intervals, at month 3, 6, 12, 18, 24, 30 and 36.

In still a further embodiment, the immunotherapeutic agent, particularly recombinant BCG ΔUrec::Hly+, is used for the treatment of non-muscle invasive bladder cancer in patients with recurrence after standard BCG therapy. The immunotherapeutic agent is administered into the bladder according to a schedule involving weekly instillations during an induction phase with e.g. 6 weekly instillations, a first maintenance phase after about 3 months with e.g. 3 weekly instillations, a second maintenance phase after about 6 months with e.g. 3 instillations and a third maintenance phase after about 12 months with e.g. 3 instillations.

The administration as immunotherapeutic agent of the recombinant Mycobacterium cell to site of a solid tumor as described above, may be combined with further anti-tumor therapy, e.g., radiation and/or chemotherapy. Further, the immunotherapy as described above, may be combined with a non-tumor site specific administration of the recombinant Mycobacterium cell in order to provide a general stimulation of the immune system. This non-site specific administration may be effected as described in WO 2012/085101, e.g. before surgery of the primary tumor. In this case, the agent is preferably administered to a human subject in a dose of about 1-10×10⁵, preferably about 2-8×10⁵ cells. The agent is preferably administered as a single dose, e.g., by injection. Subcutaneous injection is preferred. Further it is preferred to administer the agent without adjuvant.

Further, the invention is described in more detail by the following Figures and Examples 1-3.

The immunotherapeutic agent “rBCG” used in these examples is recombinant M. bovis (BCG) Danish subtype Prague with an inactivated ureC sequence (ΔUrec) and without functional selection marker gene which expresses an Ag85B/Hly fusion protein as shown in SEQ ID No.2 (Hly+).

### Example 1 Single Dose Toxicity Study of Recombinant BCG (rBCG) in Rats Following Intravesicular Instillation

| 1.1 Conduct of study | | |
|---|---|---|
| Test item | rBCG lyophilized | |
| | (rBCG Danish subtype Prague ΔUrec::Hly+ | |
| | w/o functional selection marker gene) | |
| Approximate viable counts | 5.41 × 10⁸ CFU/vial | |
| Reference item | BCG medac | |
| Approximate viable counts | 2 × 10⁸ to 2 × 10⁸ CFU/vial | |
| Test species/Strain/Stock | Rat/CD ®/Crl:CD(SD) | |
| Breeder | Charles River Laboratories, Research | |
| | Models, and Services, Germany GmbH | |
| | Sandhofer Weg 7 | |
| | 97633 Sulzfeld, Germany | |
| Number and sex of animals | 23 female animals; | |
| | 3 animals for group 1; | |
| | 5 animals for groups 2 to 5. | |
| Dose regime | Group 1: | Control (diluent) |
| | Group 2: | ~2 × 10⁸ CFU rBCG |
| | | (Iyophilized)Ianimal |
| | Group 3: | ~2 × 10⁸ CFU rBCG |
| | | (Iyophilized)Ianimal |
| | Group 4: | ~2 × 10⁶ CFU rBCG |
| | | (frozen w/o cryoprotectant)/ |
| | | animal |
| | Group 5: | ~2 × 10⁶ CFU BCG |
| | | medac/animal |

| | |
|---|---|
| Route of administration | Intravesical instillation in the bladder |
| Frequency of administration | Single dose on test day 1. |
| Administration volume | 500 μL/animal |
| Duration of study | 12 adaptation days |
| | 4 in-life test weeks |
| | 28 incubation days |

| | |
|---|---|
| 1.2 Results | |
| Mortality | None of the animals died prematurely. |
| Clinical signs | No changes of behaviour, external |
| | appearance or condition of faeces were |
| | observed for any animal at any treatment. |
| Body weight | The body weight of all animals of all dose |
| | groups was in the normal range throughout |
| | the course of the study. |
| Food and drinking | The food intake of all animals of all dose |
| water consumption | groups was in the normal range throughout |
| | the course of the study. |
| | The visual appraisal of the drinking water |
| | consumption did not reveal any test or |
| | reference item-related influence. |
| IL-2 levels | The IL-2 levels in urine and serum of all |
| | animals of all groups were below the lower |
| | limit of quantification. |
| Macroscopic post mortem | No test or reference item-related changes |
| findings | were noted. |
| Organ weights | No test or reference item-related changes |
| | were noted. |
| CFU counts | rBCG (groups 2 to 4) |
| | vs. control (group 1) |
| | No test-item related CFU counts were |
| | noted for the examined organs and the blood |
| | of the animals treated once with an |
| | intravesical instillation of 2 × 10⁶ or |
| | 2 × 10⁸ CFU rBCG (Iyophilized)/animal, or |
| | of 2 × 10⁶ CFU rBCG (frozen)/animal. In |
| | particular, no CFU counts at all were noted |
| | for the urinary bladder four weeks after |
| | instillation of the test item, indicating a rapid |
| | clearance of the administered mycobacteria |
| | from the site of instillation. |
| | No differences were noted between the |
| | animals treated with 2 × 10⁶ or 2 × 10⁸ CFU |
| | rBCG (Iyophilized)/animal or with 2 × 10⁶ |
| | CFU rBCG (frozen)/animal and the control |
| | animals. |
| | BCG medac (group 5) vs. control (group 1) |
| | No reference-item related CFU counts were |
| | noted for the examined organs and the blood |
| | of the animals treated once with an |
| | intravesical instillation of 2 × 10⁶ BCG |
| | medac/animal. In particular, no CFU counts |
| | at all were noted for the urinary bladder four |
| | weeks after instillation of the reference item, |
| | indicating a rapid clearance of the |
| | administered mycobacteria from the site of |
| | instillation. |
| | No differences were noted between the |
| | animals treated with 2 × 10⁶ BCG |
| | medac/animal and the control animals. |
| | rBCG (groups 2 to 4) |
| | vs. BCG medac (group 5) |
| | No difference in CFU counts was noted for |
| | the examined organs and the blood of the |
| | animals treated once with an intravesical |
| | instillation of 2 × 10⁶ or 2 × 10⁸ CFU rBCG |
| | (lyophilized)/animal, or of 2 × 10⁶ CFU |
| | rBCG (frozen)/animal compared to the |
| | reference group treated in the same way |
| | with 2 × 10⁶ CFU BCG medac/animal. |
| Immunohistochemistry | Immunohistochemistry for lymphocyte sub- |
| | typing in the urinary bladder tissue revealed |
| | the highest incidence of focal and multifocal |
| | lymphocytic infiltration with CD4 and CD8 |
| | positive cells in groups 2 and 3 treated once |
| | with 2 × 10⁶ or 2 × 10⁸ CFU rBCG |
| | (lyophilized)/animal by intravesical |
| | instillation, whereas animals of groups 1 |
| | (control), 4 and 5 expressed CD4 and CD8 |
| | positive lymphocytes only as single cell |
| | infiltrate levels (diffuse infiltration). Nearly all |
| | animals (12 of 13) of groups 1 (control), 4 |
| | and 5 expressed CD4 and CD8 positive |
| | lymphocytes only as single cell infiltrate |
| | levels (diffuse infiltration). In contrast nearly |
| | all animals (9 of 10) of groups 2 and 3 |
| | contained CD4 and CD8 positive |
| | lymphocytes in focal or multifocal |
| | lymphocyte infiltrates in addition to the |
| | diffuse infiltration. |
| | Neutrophilic granulocytes could rarely be |
| | detected in any slides examined. |

In conclusion, no signs of toxicity were noted for the animals treated with the test item, the reference item or the dilutent. There were no differences in the systemic spread of mycobacteria in blood and organs between rBCG and BCG medac for the treatment by intravesical instillation. No test-item related CFU counts were noted for the examined organs and the blood of the animals treated once with an intravesical instillation of rBCG compared to the control animals. In particular, no CFU counts at all were noted for the urinary bladder 4 weeks after instillation of the test item, indicating a rapid clearance of the administered mycobacteria from the site of instillation.

Immunohistochemistry for lymphocyte sub-typing in the urinary bladder tissue revealed the highest incidence of focal and multifocal lymphocytic infiltration with CD4 and CD8 positive cells in groups 2 and 3 treated once with 2×10⁶ or 2×10⁸ CFU rBCG/animal by intravesical instillation, whereas animals of groups 1 (control), 4 and 5 expressed CD4 and CD8 positive lymphocytes only as single cell infiltrate levels (diffuse infiltration). Nearly all animals (12 of 13) of groups 1 (control), 4 and 5 expressed CD4 and CD8 positive lymphocytes only as single cell infiltrate levels (diffuse infiltration). In contrast, nearly all animals (9 of 10) of groups 2 and 3 contained CD4 and CD8 positive lymphocytes in focal or multifocal lymphocyte infiltrates in addition to the diffuse infiltration. FIG. 3A shows focal and multifocal lymphocytic infiltration after administration of rBCG. FIG. 3B shows only diffuse and singular infiltration after administration of BCG medac (200× magnification).

### Example 2 Repeated Dose Toxicity Study of Recombinant BCG (rBCG) in Rats Following Intravesicular Instillation

| 2.1 Conduct of study | | |
|---|---|---|
| Test item | rBCG lyophilized | |
| | (rBCG Danish subtype Prague ΔUrec::Hly+ | |
| | w/o functional selection marker gene) | |
| Approximate viable counts | 5.41 × 10⁸ CFU/vial | |
| Reference item | BCG medac | |
| Approximate viable counts | 2 × 10⁸ to 2 × 10⁹ CFU/vial | |
| Test species/Strain/Stock | Rat/CD ®/Crl:CD(SD) | |
| Breeder | Charles River Laboratories, Research | |
| | Models, and Services, Germany GmbH | |
| | Sandhofer Weg 7 | |
| | 97633 Sulzfeld, Germany | |
| Number and sex of animals | 23 female animals; | |
| | 3 animals for group 1; | |
| | 5 animals for groups 2 to 5. | |
| Dose regime | Group 1: | Control (diluent) |
| | Group 2: | ~2 × 10⁶ CFU rBCG |
| | | (lyophilized)/animal |
| | Group 3: | ~2 × 10⁸ CFU rBCG |
| | | (lyophilized)/animal |
| | Group 4: | ~2 × 10⁶ CFU rBCG |
| | | (frozen w/o cryoprotectant)/ |
| | | animal |
| | Group 5: | ~2 × 10⁶ CFU BCG medac/ |
| | | animal |

| | |
|---|---|
| Route of administration | Intravesical instillation in the bladder |
| Frequency of administration | Repeated administration; once weekly on |
| | test days 1, 8, 15, 22, 29, and 36. |
| Administration volume | 500 μL/animal |
| Duration of study | 21 adaptation days |
| | 9 in-life test weeks |
| | 28 incubation days |
| 2.2 Results | |
| Mortality | None of the animals died prematurely. |
| Clinical signs | No changes of behaviour, external |
| | appearance or condition of faeces were |
| | observed for any animal at any treatment. |
| Body weight | The body weight of all animals of all dose |
| | groups was in the normal range throughout |
| | the course of the study. |
| Food and drinking | The food intake of all animals of all dose |
| water consumption | groups was in the normal range throughout |
| | the course of the study. |
| | The visual appraisal of the drinking water |
| | consumption did not reveal any test or |
| | reference item-related influence. |
| IL-2 levels | The IL-2 levels in urine and serum of all |
| | animals of all groups were below the lower |
| | limit of quantification. |
| Delayed type hyper- | No delayed type hypersensitivity was noted. |
| sensitivity (DTH assay) | |
| Macroscopic post mortem | No test or reference item-related changes |
| findings | were noted. |
| Organ weights | No test or reference item-related changes |
| | were noted. |
| CFU counts | rBCG (groups 2 to 4) |
| | vs. control (group 1) |
| | No test-item related CFU counts were noted |
| | for the examined organs and the blood of the |
| | animals treated six times with an intravesical |
| | instillation of 2 × 10⁶ or 2 × 10⁸ CFU rBCG |
| | (Iyophilized)/animal, or of 2 × 10⁸ CFU |
| | rBCG (frozen/w/o cryoprotectant)/animal. |
| | In particular, no CFU counts at all were |
| | noted for the urinary bladder four weeks after |
| | the last instillation of the test item, indicating |
| | a rapid clearance of the administered |
| | mycobacteria from the site of instillation. |
| | No differences were noted between the |
| | animals treated with 2 × 10⁶ or 2 × 10⁸ CFU |
| | rBCG (Iyophilized)/animal or with 2 × 10⁸ |
| | CFU rBCG (frozen)/animal and the control |
| | animals. |
| | BCG medac (group 5) vs. control (group 1) |
| | No reference-item related CFU counts were |
| | noted for the examined organs and the blood |
| | of the animals treated six times with an |
| | intravesical instillation of 2 × 10⁶ BCG |
| | medac/animal. In particular, no CFU counts |
| | at all were noted for the urinary bladder four |
| | weeks after the last instillation of the |
| | reference item, indicating a rapid clearance |
| | of the administered mycobacteria from the |
| | site of instillation. |
| | No differences were noted between the |
| | animals treated with 2 × 10⁶ BCG |
| | medac/animal and the control animals. |
| | 3 rBCG (groups 2 to 4) |
| | vs. BCG medac (group 5) |
| | No difference in CFU counts was noted for |
| | the examined organs and the blood of the |
| | animals treated six times with an intravesical |
| | instillation of 2 × 10⁶ or 2 × 10⁸ CFU rBCG |
| | (lyophilized)/ animal, or of 2 × 10⁶ CFU |
| | rBCG (frozen)/ animal compared to the |
| | reference group treated in the same way |
| | with 2 × 10⁶ CFU BCG medac/animal. |
| Immunohistochemistry | Immunohistochemistry for lymphocyte sub- |
| | typing in the urinary bladder tissue revealed |
| | the highest incidence of multifocal |
| | lymphocytic infiltration with CD4 and CD8 |
| | positive cells in group 2 treated with |
| | 2 × 10⁶ CFU rBCG/animal by 6 intravesical |
| | instillations, followed by group 3 treated with |
| | 2 × 10⁸ CFU rBCG/animal by 6 intravesical |
| | instillations, whereas animals of groups 1 |
| | (control), 4 and 5 expressed CD4 and CD8 |
| | positive lymphocytes only as single cell |
| | infiltrate levels (diffuse infiltration). |
| | Neutrophilic granulocytes could rarely be |
| | detected in any slides examined. |

In conclusion, no signs of toxicity were noted for the animals treated with the test item, the reference item or the diluent. There were no differences in the systemic spread of mycobacteria in blood and organs between rBCG and BCG medac for the treatment by intravesical instillation. No test-item related CFU counts were noted for the examined organs and the blood of the animals treated six times with an intravesical instillation of rBCG compared to the control animals. In particular, no CFU counts at all were noted for the urinary bladder 4 weeks after the last instillation of the test item, indicating a rapid clearance of the administered mycobacteria from the site of instillation.

Immunohistochemistry for lymphocyte sub-typing in the urinary bladder tissue revealed the highest incidence of multifocal lymphocytic infiltration with CD4 and CD8 positive cells in group 2 treated with 2×10⁶ CFU rBCG/animal by 6 intravesical instillations, followed by group 3 treated with 2×10⁸ CFU rBCG/animal by 6 intravesical instillations, whereas animals of groups 1 (control), 4 and 5 expressed CD4 and CD8 positive lymphocytes only as single cell infiltrate levels (diffuse infiltration). FIG. 4A shows focal and multifocal lymphocytic infiltration after administration of rBCG. FIG. 4B shows only diffuse and singular infiltration after administration of BCG medac (200× magnification).

### Example 3

Phase I/II Single Arm Clinical Trial of Recombinant Bacillus Calmette-Guerin (rBCG) Immunotherapy in Non-Muscle-Invasive Bladder Cancer Recurrence after Conventional BCG Therapy

### Patients and Methods

### Study Design and Participants

The trial was designed as a multicentre, open-label, single-arm, phase I/II study and conducted in compliance with the current version of the declaration of Helsinki, the ICH-GCP, and with national legal and regulatory requirements. Written informed consent was obtained from all patients prior to enrolment.

Eligible patients had recurrent NMIBC with intermediate and high-risk for progression (score 7-23 based on the European Organization for Research and Treatment of Cancer scoring system) after conventional BCG therapy.

Persistent T1 disease or high-grade non-muscle invasive tumour(s) present at both 3 and 6 months after start of the previous cycle of BCG therapy, or any worsening of the disease under BCG treatment, such as a higher number of recurrences, higher tumour category or higher grade, or appearance of carcinoma in situ (CIS), in spite of an initial response were required (EAU 2008 guideline definition). Prior therapy was defined as one previous cycle of intravesical BCG (induction phase≥5 instillations ±BCG maintenance). For inclusion, histologically confirmed diagnosis of recurrent NMIBC and a repeat transurethral resection of the bladder (TURB) confirming a tumour-free state according to current pathology guidelines were mandatory. In patients presenting with CIS, selective upper tract cytology and biopsies of the prostatic urethra were recommended. At the start of the study treatment, bladder wash cytology had to be negative, except for patients with pure or concomitant CIS and imaging without evidence of metastatic disease.

Exclusion criteria were stage T2 urothelial carcinoma of the bladder, concomitant urothelial carcinoma of the upper urinary tract, the non-prostatic urethra, or evidence of metastatic disease (https://clinicaltrials.gov/ct/show/NCT02371447).

rBCG was provided as a formulated lyophilised powder of 1-19.2×10E8 CFU/vial of live Mycobacterium bovis BCGΔureC::hly (SIIPL, Serum Institute of India Private Limited, Pune, India) and reconstituted in 50 ml of 0.9% saline water for intravesical application. Patients were scheduled for standard treatment of 6 weekly intravesical instillations followed by maintenance of 1 year (3 instillations at 3, 6 and 12 months after first instillation).

The primary endpoint of the phase II part was defined as the recurrence-free rate (RFR) in the bladder 60 weeks after registration. In case of suspected recurrence due to positive cytology or visual detection of a tumour in the bladder at cystoscopy, histological proof of the recurrence was required. A recurrence in the bladder associated with cancer in the prostatic urethra or evidence for cancer in the upper urinary tract were not considered as a recurrence in the bladder. Predefined secondary endpoints included time from trial registration to recurrence in the bladder, time to progression, overall survival (OS), AEs, tolerability, and QoL. Progression was defined as progression to muscle-invasive bladder cancer or progression to metastatic disease. OS was calculated from registration until death from any cause. Tolerability was defined as the proportion of patients finishing five instillations of induction within 12 weeks after treatment start.

### Statistical Analyses

For sample size calculation, we estimated the RFR from one of the few prospective randomized European trials comparing BCG re-exposure versus gemcitabine. In this trial, the group receiving BCG had an estimated RFR and progression-free rate 60 weeks after the last TURB of 15% and 62.5%, respectively. A single-stage design was used with H0: RFR at 60 weeks≤15%; and H1: RFR at 60 weeks≥30%. For a significance level of 10% and a power of 80%, 37 evaluable patients for phase II were required based on the Kaplan-Meier estimator evaluated 60 weeks after registration (R package Optlnterim). The 6 patients from the phase I part were included for the phase II part. To account for non-evaluable patients, the sample size was increased to 39 patients.

### Results

### Patients

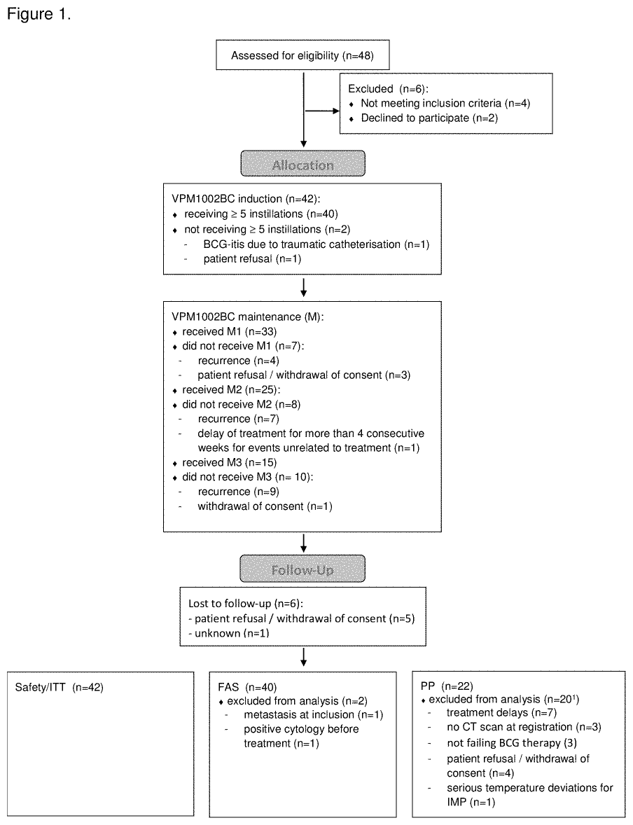

From Sep. 2015 to Apr. 2018, 42 patients were enrolled into the trial. A total of 16 sites were activated and 14 sites actively recruited patients. Two patients had to be excluded from efficacy analysis because of major inclusion criteria violation but were kept in for safety/ITT analysis. Forty patients defined the full analysis set (FAS) and the per protocol population (PP) consisted of 22 patients. Definitions of analysed populations and detailed reasons for exclusions are given in FIG. 1.

The detailed tumour characteristics of the primary NMIBC occurrences of the patient cohort are given in Table 1 and the detailed patient and tumour characteristics of the FAS in Table 2. The median risk for progression of the recurrent NMIBC increased by 7 risk points as compared to the primary occurrence. A history of chemical exposure as a risk factor for bladder cancer was identified in 3 patients, while 21 patients (52.5%) had a smoking history with a median (range) of 38 (4.0-99.0) pack-years. Concomitant or pure CIS was present in 27 (67.5%) patients. Fourteen patients (35%) received BCG maintenance therapy. The median progression score was 16 (7-20) for those patients with and 16 (7-19) for those without previous BCG maintenance.

All patients enrolled in the trial started trial treatment. Thirty-two patients (80%) started maintenance therapy. Fifteen patients (37.5%) received all scheduled instillations of induction and maintenance therapy. Detailed reasons for stopping treatment during the trial are given in FIG. 1. Median follow-up at the time of analysis was 74.4 (12.3-140.9) weeks.

### Primary Endpoint

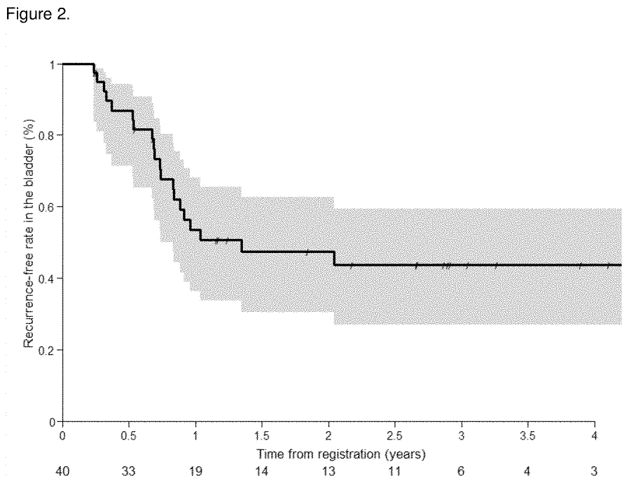

After rBCG induction and maintenance therapy, 49.3% [95% Cl 32.1, 64.4] of the patients were without evidence of recurrence in the bladder 60 weeks after trial registration. After a median follow-up of 2.9 years, the RFR remained at 47.4% [30.4%, 62.6%] and 43.7% [26.9%, 59.4%] 2 and 3 years after trial registration, respectively. The median time from trial registration to recurrence in the bladder was 54.1 weeks [95% Cl 38.4 weeks] (FIG. 2). The RFR in the bladder for the ITT and PP populations 60 weeks after registration was 48.0% [95% Cl 31.1, 63.0] and 40.8% [95% Cl 19.9, 60.9], respectively. If accounting for all potential recurrences including histologically unconfirmed but clinically evident extravesical recurrences in the FAS population, the 60-week RFR reached 47.6% [95% Cl 30.8, 62.6]. Out of the patients with CIS (n=27), 15 (55.6%; 95% Cl [35.3%,74.5%]) had a complete response (CR) at 12 weeks after treatment start. The median duration of response (DOR) for these 15 patients was 1.1 years (95% Cl [0.4 years—not reached]). Further subgroup evaluations related to the primary endpoint are given in Table 2.

### Secondary Endpoints

Seven patients from the FAS experienced progressive disease (median time to progression not reached). Three patients showed progression to muscle-invasive disease, and 4 patients progressed with metastatic disease (regional lymph nodes n=3, distant metastasis n=1). None of the patients with metastatic disease were found to have concomitant muscle invasive disease in the bladder.

Follow-up treatment for the patients with recurrence after trial treatment is listed in Table 4.

Until the time of analysis, 5 patients had died (2 from bladder cancer, 2 from other cancers not known at inclusion, and one from acute respiratory distress syndrome unrelated to the study treatment).

In summary, 60 weeks after trial registration, progression-free rate and OS rate were 76.3% [95% Cl 56.4, 88.0] and 92.9% [95% Cl 74.3, 98.2], respectively.

### Adverse Events and Tolerability

Treatment-related AEs are listed in Table 5. The major grade 2 AE term was genitourinary tract infection with common uropathogenic bacteria in one third of the patients. Two patients had to undergo in-hospital antibiotic treatment for urogenital tract infection.

No grade 4 or 5 AEs occurred during the treatment phase. Two of the 42 patients did receive less than five instillations, one because of a BCG-induced systemic inflammatory reaction (BCGitis) due to traumatic catheterisation and one because of patient refusal (FIG. 1).

### PPD Testing

77.5% of the patients were PPD negative (PPD−) at treatment start (Table 1). Conversion of the PPD test after finishing the rBCG therapy was seen in 7 patients only (PPD- to PPD+: n=5; PPD+ to PPD−: n=2). Fifteen patients (37.5%) did not convert from PPD- to PPD+. In these patients, the RFR in the bladder was 62.9% (Table 2).

### Quality of Life

All 40 patients in the FAS completed the QoL questionnaire at baseline; 32 of 33 (97%) patients who started the maintenance therapy completed it at the beginning of maintenance therapy and 32 of 40 (77.5%) completed it at the end of treatment. Fourteen patients (45.2%) completed the QoL assessment after the 15 instillations as required by protocol. Patients reported high levels of QoL for the functional scales and a low symptom burden at baseline. Some impairment was reported with regard to urinary symptoms, future worries, and sexual issues. Most of the QoL scales showed stable scores from baseline to the beginning of maintenance therapy. Small improvements were observed for emotional functioning and urinary symptoms. Future worries improved (i.e., patients expressed less worries) to a clinically relevant extent. Almost half (n=13, 49%) of the patients reported a clinically relevant improvement in emotional functioning, whereas approx. one third reported a clinically relevant deterioration in physical well-being (n=10, 30%), global health status/QoL (n=11, 33%), or fatigue (n=10, 30%) during induction therapy. No major changes from baseline to the end of treatment were observed for any of the QoL scales in those patients who completed treatment according to protocol.

### DISCUSSION

We have chosen a high-risk population for recurrence and progression with previous conventional BCG exposure to test rBCG. Given the poor outcome of BCG re-exposure in these patients with reported 12 and 24 months RFR of 15% and 3%, respectively, we considered a placebo-controlled trial or a trial comparator with conventional BCG as unethical. With 49.3% RFR in the bladder, this single-arm trial has clearly met the primary endpoint of the predefined RFR>30% 60 weeks after trial registration. This is also true when extending the definition of recurrence to extra-vesical recurrences including histologically unconfirmed clinically evident recurrences in the FAS population, the ITT and the PP population, as well as whether the patients received BCG maintenance or not (Table 2). With a RFR of 47.4% and 43.7% after 2 and 3 years, the treatment response remains stable indicating a potentially promising long-term effect.

After the initiation of this trial, the Food and Drug Administration (FDA) published a new definition for BCG failure and the International Bladder Cancer Group recommended a clinically meaningful initial complete response rate (for CIS) or recurrence-free rate (for papillary tumours) of at least 30% at 12 months for single arm trials in BCG unresponsive patients. According to the FDA definition of BCG failure, 16 patients (40.0%) qualified for BCG unresponsive disease. When applying these more stringent criteria, the 60-week RFR dropped to 24.0% (Table 2).

Further subgroup analysis related to the primary endpoint indicated that smoking status, presence of CIS, and previous BCG maintenance therapy were possibly associated with a worse outcome in this trial, which were, however, not statistically significant—a result again limited by the small sample size (Table 2).

Most of the patients with recurrence underwent cystectomy or other local treatments, and 3 patients underwent systemic chemotherapy (Table 3). While missed metastatic disease at inclusion is a potential explanation for such a fatal development, it cannot be excluded that metastatic disease evolved after TUR and/or under intravesical trial therapy. Notably, none of the patients with metastatic disease were found to have concomitant muscle invasive disease in the bladder.

### Adverse Events and Tolerability

The most common AE was genitourinary (GU) infection with common bacteria, occurring in one third of the patients. Potential explanations are i) that lubricants containing antiseptics for instillations were not allowed, and ii) that asymptomatic patients with positive urine dipstick had to undergo antibiotic treatment when urine culture was positive. Importantly, the GU infections did not significantly impact on QoL of the patients (not shown). No grade 4 or 5 AE occurred, and tolerability (defined as patients receiving more than 4 instillations during induction) was 95.2%. The treatment can therefore be considered as safe and well tolerated.

### PPD Testing

Experimental models have suggested that either a PPD+ state prior to the start of treatment or a conversion from PPD− to PPD+ state during treatment might predict response to therapy. In clinical trials, the clinical correlation of the PPD test with the outcome has been conflicted by heterogeneous results, by previous mycobacterial exposure, and different handling in the execution and interpretation of the test itself. In SAKK 06/14, most patients (77.5%) had a negative PPD test before the start of the treatment with rBCG, although they had been exposed to BCG previously. Unexpectedly, patients with negative PPD test at inclusion showed a 60-week RFR in the bladder of 52.8%, and even without conversion from PPD- to PPD+, the RFR in the bladder remained 62.9% (n=15; Table 2), indicating that a negative PPD test and non-conversion might not be able to predict treatment outcome in this setting.

### QoL

Patients reported overall stable QoL during induction and maintenance treatment, with small improvements in emotional functioning and future worries. Around 30% of patients reported clinically relevant deteriorations for physical functioning, global QoL, or fatigue during induction therapy. Interestingly, for patients who completed the treatment according to protocol no major changes were observed for any of the QoL scales. Again, the limited sample size without a comparator arm stresses for a cautious interpretation of these QoL results.

If the favorable Qol and tolerability is confirmed in larger studies, rBCG has the potential to decrease the proportion of patients becoming intolerable to BCG treatment and may be combined with other agents such as check-point inhibitors in order to increase efficiency.

### CONCLUSIONS

SAKK 06/14 has met the primary endpoint and demonstrates freedom from recurrence in the bladder after intravesical rBCG treatment in almost half of the patients after one year. rBCG has a promising tolerability, safety, and QoL profile.

### Tables and Figure Legends

Table 1. Baseline tumour characteristics of first occurrence of NMIBC (before first conventional BCG treatment).

Table 2. Baseline patient and tumour characteristics of the FAS (full analysis set). WHO: World Health Organisation. BCG: Bacillus Calmette Guerin. CIS: Carcinoma in situ. EORTC: European Organisation for Research and Treatment of Cancer.

Table 3. Subgroup efficacy analysis of the FAS showing recurrence-free rate in the bladder at 60 weeks together with 95% Cl. CIS: Carcinoma in situ. BCG: Bacillus Calmette-Guerin. FDA: Food and Drug Administration. PPD: Purified protein derivative.

Table 4. Treatment of patients with recurrence and/or progression after trial treatment. BCG: Bacillus Calmette Guerin. Some patients received more than one treatment. One of the 13 patients undergoing cystectomy received surgery due to chronic bladder infection and not because of cancer recurrence.

* one of these treatments was performed based on a recurrence later than 60 weeks after registration.

Table 5. Treatment associated adverse events (AE) in the treatment phase. BCG: Bacillus Calmette-Guerin. GU: Genito-urinary.

### BRIEF DESCRIPTION OF THE DRAWINGS

FIG. 1. CONSORT 2010 diagram of SAKK 06/14. BCG: Bacillus Calmette-Guerin. ITT: Intention to treat. FAS: Full analysis set (population). PP: Per-protocol (population).

¹ includes also the 2 exclusions from FAS.

FIG. 2: Kaplan Meier plot for time to recurrence in the bladder for the FAS (median follow up 2.9 years).

FIGS. 3A and 3B: Immunohistochemistry for lymphocyte sub-typing in the urinary bladder tissue revealed the highest incidence of focal and multifocal lymphocytic infiltration with CD4 and CD8 positive cells in groups 2 and 3 treated once with 2×106 or 2×108 CFU rBCG/animal by intravesical instillation, whereas animals of groups 1 (control), 4 and 5 expressed CD4 and CD8 positive lymphocytes only as single cell infiltrate levels (diffuse infiltration). FIG. 3A shows focal and multifocal lymphocytic infiltration after administration of rBCG. FIG. 3B shows only diffuse and singular infiltration after administration of BCG medac (200× magnification).

FIGS. 4A and 4B: Immunohistochemistry for lymphocyte sub-typing in the urinary bladder tissue revealed the highest incidence of multifocal lymphocytic infiltration with CD4 and CD8 positive cells in group 2 treated with 2×106 CFU rBCG/animal by 6 intravesical instillations, followed by group 3 treated with 2×108 CFU rBCG/animal by 6 intravesical instillations, whereas animals of groups 1 (control), 4 and 5 expressed CD4 and CD8 positive lymphocytes only as single cell infiltrate levels (diffuse infiltration). FIG. 4A shows focal and multifocal lymphocytic infiltration after administration of rBCG. FIG. 4B shows only diffuse and singular infiltration after administration of BCG medac (200× magnification).

TABLE 1

| | FAS | |
|---|---|---|
| Variable | n (%) | |
| T classification | | |
| T1 | 18 | (45.0) |
| T1/CIS | 6 | (15.0) |
| Ta | 6 | (15.0) |
| Ta/CIS | 5 | (12.5) |
| CIS | 5 | (12.5) |
| 1973 WHO grading | | |
| Grade 2 | 5 | (12.5) |
| Grade 3 | 34 | (85.0) |
| Unknown | 1 | (2.5) |
| 2004 WHO grading | | |
| Low-grade urothelial carcinoma | 4 | (10.0) |
| High-grade urothelial carcinoma | 35 | (87.5) |
| Unknown | 1 | (2.5) |
| EORTC progression score | | |
| 0 | 1 | (2.5) |
| 1-6 | 7 | (17.5) |
| 7-13 | 26 | (65.0) |
| 14-23 | 6 | (15.0) |
| Median (min, max) | 9 | (0.0 -18) |
| EORTC recurrence | | |
| 1-4 | 35 | (87.5) |
| 5-9 | 5 | (12.5) |
| Median (min, max) | 3 | (1-7) |

TABLE 2

| | FAS | | |
|---|---|---|---|
| Variable | n (%) | | |
| Sex | | | |
| Female | 4 | (10.0) | |
| Male | 36 | (90.0) | |
| WHO performance score | | | |
| 0 | 35 | (87.5) | |
| 1 | 5 | (12.5) | |
| Smoking status | | | |
| Current smoker | 10 | (25.0) | |
| Former smoker | 11 | (27.5) | |
| Non-smoker | 18 | (45.0) | |
| Not available | 1 | (2.5) | |
| BCG maintenance performed during previous therapy | | | |
| No | 26 | (65.0) | |
| Yes | 14 | (35.0) | |
| T classification for recurrence | | | |
| T1 | 7 | (17.5) | |
| T1/CIS | 5 | (12.5) | |
| Ta | 6 | (15.0) | |
| Ta/CIS | 5 | (12.5) | |
| CIS | 17 | (42.5) | |
| 1973 WHO grading of recurrence | | | |
| Grade 2 | 3 | (7.5) | |
| Grade 3 | 37 | (92.5) | |
| 2004 WHO grading of recurrence for study inclusion | | | |
| High-grade urothelial carcinoma | 40 | (100.0) | |
| EORTC progression score | | | |
| 7-13 | 12 | (30.0) | |
| 14-23 | 28 | (70.0) | |
| Median (min, max) | 16 | (7, 20) | |
| EORTC recurrence score | | | |
| 1-4 | 4 | (10.0) | |
| 5-9 | 30 | (75.0) | |
| 10-17 | 6 | (15.0) | |
| Median (min, max) | 8 | (4, 11) | |
| Baseline PPD test | | | |
| No reaction | 31 | (77.5%) | |
| Positive | 7 | (17.5%) | |
| Missing | 2 | | (5.0%) |

TABLE 3

| | | | |
|---|---|---|---|
| | no | yes/former | |
| Smoking | (n = 18) | (n = 21) | p |
| Recurrence-free rate in | 53.1 | 45.5 | 0.652 |
| the bladder at 60 weeks | [27.8, 73.2] | [22.4, 66.1] | |
| in % [95% CI] | | | |
| | no | yes | |
| CIS | (n = 13) | (n = 27) | p |
| Recurrence-free rate in | 61.5 | 42.4 | 0.289 |
| the bladder at 60 weeks | [30.8, 81.8] | [22.0, 61.5] | |
| in % [95% CI] | | | |
| Previous BCG | no | yes | |
| maintenance | (n = 26) | (n = 14) | p |
| Recurrence-free rate in | 54.3 | 38.4 | 0.371 |
| the bladder at 60 weeks | [32.9, 71.6] | [12.2, 64.6] | |
| in % [95% CI] | | | |
| BCG unresponsive | no | yes | |
| (FDA definition) | (n = 24) | (n = 16) | p |
| Recurrence-free rate in | 63.6% | 24.0% | 0.022 |
| the bladder at 60 weeks | [40.3%, 79.9%] | [5.9%, 48.8%] | |
| in % [95% CI] | | | |
| | no | PPD− to PPD+ | |
| PPD conversion | (n = 15) | (n = 5) | p |
| Recurrence-free rate in | 62.9% | 80.0% | 0.505 |
| the bladder at 60 weeks | [32.3%, 82.6%] | [20.4%, 96.9%] | |
| in % [95% CI] | | | |

TABLE 4

| Treatment | n (%) | |
|---|---|---|
| Cystectomy | 13* | (32.5) |
| BCG (upper tract) | 2 | (5.0) |
| Systemic chemotherapy | 3 | (7.5) |
| Intravesical chemotherapy | 6* | (15.0) |

TABLE 5

| | Grade 1 | Grade 2 | Grade 3 |
|---|---|---|---|
| AE | n (%) | n (%) | n (%) |
| Vertigo | | 1 (2.4) | |
| Gastrointestinal disorders | 2 (4.8) | | |
| Fatigue | 2 (4.8) | 3 (7.1) | |
| Fever | 2 (4.8) | 1 (2.4) | |
| Frequency, urgency | 7 (16.7) | 5 (11.9) | |
| Malaise | | 1 (2.4) | |
| BCG induced systemic inflammatory | | 1 (2.4) | |
| reaction | | | |
| Cold | | 1 (2.4) | |
| GU infection | | 14 (33.3) | 2 (4.8) |
| Alanine aminotransferase increased | | 1 (2.4) | |
| Neuralgia | | 1 (2.4) | |
| Hematuria | 2 (4.8) | | |
| Macrohematuria | 1 (2.4) | | |
| Urinary retention | 1 (2.4) | | |
| Urinary tract obstruction | 1 (2.4) | 1 (2.4) | |
| Urinary tract pain | 6 (14.3) | 1 (2.4) | |
| Vaginal pain | | 1 (2.4) | |
| Skin affection | 3 (7.1) | 2 (4.8) | |
| Thromboembolic event | | 1 (2.4) | |

1. A recombinant Mycobacterium bovis cell which comprises a recombinant nucleic acid molecule encoding a fusion polypeptide comprising:
(a) a domain capable of eliciting an immune response, and
(b) a Listeria phagolysosomal escape domain
in combination with a carrier suitable for use with an immunotherapeutic agent in the treatment of bladder carcinoma, in particular recurrent bladder carcinoma, wherein the individual to be treated has relapsed and/or has progressed after a first treatment for bladder carcinoma.

2. The cell of claim 1, wherein said cell is a urease-deficient cell.

3. The cell of claim 1, wherein said cell is a recombinant M. bovis BCG cell from strain Danish subtype Prague.

4. The cell of claim 1, wherein the domain capable of eliciting an immune response is selected from the group consisting of immunogenic peptides and polypeptides from M. bovis or M. tuberculosis.

5. The cell of claim 1, wherein the recombinant nucleic acid molecule does not comprise any functional selection marker.

6. The cell of claim 1, wherein the fusion polypeptide comprises
(a) a domain capable of eliciting an immune response comprising the amino acid sequence from aa.41 to aa.51 in SEQ ID NO: 2, and
(b) a Listeria phagolysosomal escape domain encoded by a nucleic acid molecule selected from
(i) a nucleotide sequence comprising nucleotides 211-1722 as shown in SEQ ID NO: 1,
(ii) a nucleotide sequence which encodes the same amino acid sequence as the sequence from (i), and
(iii) a nucleotide sequence hybridising under stringent conditions with the sequence from (i) or (ii).

7. The cell of claim 1, wherein the carrier is suitable for local administration of the immunotherapeutic agent to the tumor site, particularly after surgery.

8. The cell of claim 1, wherein the carrier is suitable for vesicular instillation into the urinary bladder.

9. A method for the immunotherapy of bladder carcinoma in a subject in need thereof, comprising administering to said subject a recombinant Mycobacterium bovis cell comprising a recombinant nucleic acid molecule encoding a fusion polypeptide comprising:
(a) a domain capable of eliciting an immune response, and
(b) a Listeria phagolysosomal escape domain,
wherein the subject to be treated has relapsed and/or has progressed after a first treatment for bladder carcinoma.

10. The method of claim 9, wherein said immunotherapy of bladder carcinoma produces focal and/or multifocal lymphocytic infiltration at the site of administration.

11. The method of claim 10, wherein said immunotherapy of bladder carcinoma produces for focal and/or multifocal tissue infiltration with CD4 and CD8 T cells.

12. The method of claim 10, wherein the focal and/or multifocal lymphocytic tissue infiltration is increased as compared to administration of native BCG.

13. The method of claim 9, wherein the recombinant Mycobacterium bovis cell is administered into the bladder according to a schedule involving weekly instillations during (i) an induction phase with, in particular 6 weekly instillations, (ii) a maintenance phase of at least one year, in particular a first maintenance phase after about 3 months with e.g. 3 weekly instillations, a second maintenance phase after about 6 months with e.g. 3 instillations and a third maintenance phase after about 12 months with e.g. 3 instillations.

14. The method of claim 9, wherein the recombinant Mycobacterium bovis cell is used at a dose of from about 10⁶ to 10¹⁰ CFU per administration.

15. The method of claim 9, further comprising a non-tumor site specific administration of the recombinant Mycobacterium bovis cell.

16. The method of claim 9, wherein the bladder carcinoma is non-invasive bladder carcinoma, particularly carcinoma in situ (T_{cis}), non-invasive papillary carcinoma (Tₐ), or a tumor invading subepithelial connective tissue (T₁).

17. The method of claim 9, wherein the subject to be treated has received a first treatment for bladder carcinoma selected from the group consisting of cisplatin-based chemotherapy, cisplatin-based chemotherapy followed by surgical removal of the bladder or radiation therapy, concomitant chemotherapy and standard BCG.

18. The method of claim 17, wherein the subject to be treated has received standard BCG as a first treatment for bladder carcinoma.

19. The method of claim 9, wherein the subject to be treated is a smoker.

20. The method of claim 9, wherein the subject underwent cystectomy or another local treatment or systemic chemotherapy.

21. The method of claim 9, wherein the bladder carcinoma to be treated is non-muscle-invasive bladder cancer (NMIBC).
